(19) United States
(12) Patent Application Publication
(10) Pub. No.: US 2026/0249075 A1
(43) Pub. Date: Aug. 27, 2026

(54) Muscle stimulation wristwatch

(71) Applicant: THE UNIVERSITY OF CHICAGO, Chicago, IL (US)

(72) Inventors: Pedro Lopes, Chicago, IL (US); Akifumi Takahashi, Chicago, IL (US); Yudai Tanaka, Chicago, IL (US)

(21) Appl. No.: 19/550,617

(22) Filed: Feb. 26, 2026

Related U.S. Application Data
(60) Provisional application No. 63/764,325, filed on Feb. 27, 2025.

(51) Int. Cl. A61N 1/04; A61N 1/36

(52) U.S. Cl. CPC A61N 1/0484; A61N 1/0456; A61N 1/36014

(57) ABSTRACT

Systems and methods are provided to efficiently, reliably, and safely stimulate muscle fibers controlling the wrist and hand using a device that includes an array of electrodes encircling the arm proximate to the wrist. By controlling the pattern of injection/sinking of current through the electrodes, muscle fibers in specific targeted muscles can be reliably stimulated in a manner significant enough to move the hand/wrist for a variety of applications (e.g., instruction, haptic feedback, rehabilitative training, prosthetics). Current is injected (or sunk) through a single one of the electrodes, with opposite electrodes being used as return electrodes; one or more electrodes flanking the first electrode are not used to source or sink current. Such a pattern of current injection allows muscle fibers in the wrist to be reliably recruited across a range of wrist angles and using low levels of current and voltage.

### CROSS-REFERENCE TO RELATED APPLICATIONS

This application calls priority to U.S. provisional application no. 63/764,325, filed Feb. 27, 2025, the contents of which are hereby incorporated by reference.

### BACKGROUND

It is desirable to provide a human-computer interface (HCl) that is able to provide outputs to and receive inputs from users while being minimally intrusive, so that a user can engage with their environment normally (e.g., using their hands) while also using the HCl and without the presence of the HCl interfering with their interaction with the environment. For example, electrical stimulation, applied via electrodes disposed on the skin surface, can be used to stimulate afferent nerve fibers, efferent nerve fibers, muscle fibers, or other tissues in order to directly evoke a haptic perception, to indirectly evoke a haptic perception by stimulating muscle fibers (thereby causing the fibers to contract and inducing a motion that can be perceived), to induce a desired motor movement, and/or or to accomplish some other objective. To accomplish such stimulation in a repeatable, reliable, and convenient manner, the electrodes can be applied to relevant areas of the skin (e.g., above the motor point of a target muscle to be stimulated). Such application can include individually applying the electrodes and then securing them using tape, glue, or some other means, or putting on a garment or other items configured to mount to a body part and to maintain one or more electrodes in appropriate locations on the skin (e.g., an upper armband having disposed on an inner surface thereof electrodes configured to provide stimulation to muscle of the upper arm).

### SUMMARY

In a one aspect, a system is provided that includes: (i) a plurality of electrodes; (ii) a band, wherein the band is configured to maintain the plurality of electrodes in contact with skin of a wrist of a wearer such that each electrode of the plurality of electrodes contacts the wrist at a respective different location around the wrist; and (iii) a stimulator, wherein the stimulator is configured to: (a) stimulate a first set of muscle fibers in the wrist by injecting current through a first electrode of the plurality of electrodes and using a first set of the plurality of electrodes as return electrodes, wherein the first set of electrodes comprises at least two electrodes, and (b) stimulate a second set of muscle fibers in the wrist by injecting current through a second electrode of the plurality of electrodes and using a second set of the plurality of electrodes as return electrodes, wherein the second set of electrodes comprises at least two electrodes, and wherein the second electrode differs from the first electrode.

In another aspect, a method is provided that includes: (i) applying, to a wrist of a user, a device that comprises a plurality of electrodes such that each electrode of the plurality of electrodes contacts the wrist at a respective different location around the wrist; (ii) stimulating a first set of muscle fibers in the wrist by injecting current through a first electrode of the plurality of electrodes and using a first set of the plurality of electrodes as return electrodes, wherein the first set of electrodes comprises at least two electrodes; and (iii) stimulating a second set of muscle fibers in the wrist by injecting current through a second electrode of the plurality of electrodes and using a second set of the plurality of electrodes as return electrodes, wherein the second set of electrodes comprises at least two electrodes, and wherein the second electrode differs from the first electrode.

In yet another aspect, a non-transitory computer readable medium is provided having stored thereon program instructions executable by at least one processor to cause the at least one processor to perform the above method.

In a still further aspect, system is provided that includes: (i) a controller comprising one or more processor, and (ii) a non-transitory computer readable medium having stored thereon program instructions executable by the controller to cause the controller to perform the above method.

### BRIEF DESCRIPTION OF THE DRAWINGS

The accompanying drawings are included to provide a further understanding of the system and methods of the disclosure and are incorporated in and constitute a part of this specification. The drawings illustrate one or more embodiment(s) of the disclosure, and together with the description serve to explain the principles and operation of the disclosure.

FIG. 1A depicts aspects of a system as worn on an arm, in accordance with example embodiments.

FIG. 1B depicts aspects of a system as worn on an arm, in accordance with example embodiments.

FIG. 1C depicts electrodes disposed on an arm, in accordance with example embodiments.

FIG. 1D depicts electrodes disposed on an arm, in accordance with example embodiments.

FIG. 2 depicts aspects of a system as worn on an arm that is depicted in cross-section, in accordance with example embodiments.

FIG. 3 depicts aspects of an example system.

FIG. 4 depicts aspects of an example method.

FIG. 5A depicts aspects of an experimental system.

FIG. 5B depicts aspects of an experimental system.

FIG. 5C depicts aspects of an experimental system.

FIG. 5D depicts aspects of an experimental system.

FIG. 5E depicts aspects of an experimental system.

FIG. 6 depicts aspects of an experiment.

FIG. 7 depicts aspects of an experiment.

FIG. 8 depicts aspects of an experiment.

FIG. 9 depicts aspects of a device calibration user interface and method, in accordance with example embodiments.

### DETAILED DESCRIPTION

The following detailed description describes various features and functions of the disclosed embodiments with reference to the accompanying figures. The illustrative embodiments described herein are not meant to be limiting. It may be readily understood that certain aspects of the disclosed embodiments can be arranged and combined in a wide variety of different configurations, all of which are contemplated herein.

### I. Overview

It is desirable in a variety of applications to stimulate various muscles of the body, including muscles of the arm, wrist, and/or hand. For example, such stimulation could form the basis of a haptic user interface that allows a user to receive output information (indicated in the form of motions of the hands or other sensations resulting from stimulation of the muscles) from a computer system while leaving the user's hands, legs, eyes, and other senses/body parts free to interact with the user's environment unimpeded. In practice, this is difficult to accomplish in a manner that is convenient, reliable, and low-effort. This is because prior muscular stimulation systems targeting the muscles of the wrist/hand have stimulated the muscles of the forearm via electrodes disposed on the middle or upper forearm. While electrodes located in these locations can effectively and efficiently stimulate muscle fibers, the application, calibration, use, and removal of such electrodes (e.g., individually, or as part of a garment or other wearable apparatus) is effortful and may be uncomfortable and/or cosmetically or socially undesirable.

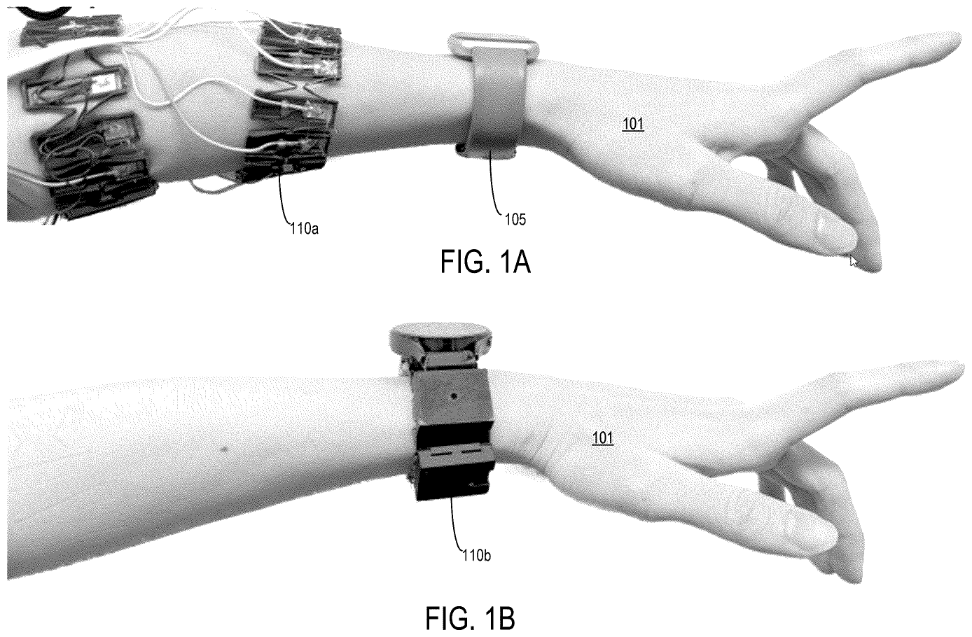

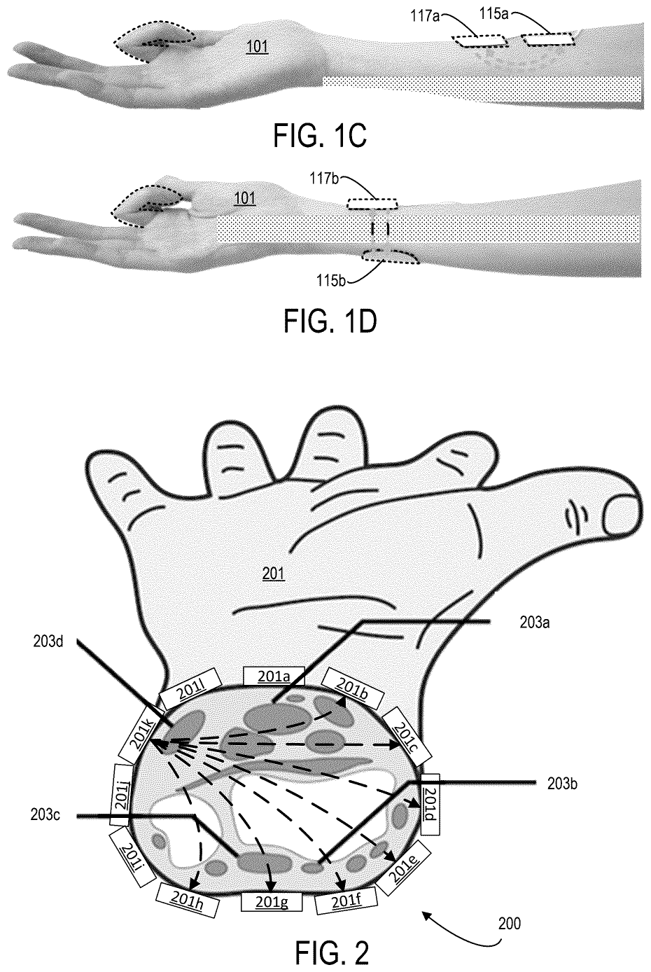

Such forearm-based muscle stimulation systems apply electrodes to the forearm (e.g., upper forearm) with significant distances, along the long axis of the forearm, between pairs of electrodes used to stimulate each muscle (e.g., multiple centimeters). FIG. 1A depicts a conventional stimulator 110a, with multiple sets of electrodes in contact with skin of the upper forearm of an arm 101 in respective locations along (and around) the forearm (also depicted is a smartwatch 105 worn on a wrist of the arm 101). FIG. 1C depicts the use of such a system to stimulate a particular muscle (flexor indicis longus, as shown in FIG. 1C), and thereby cause motion of the wrist and/or hand (flexion of the index finger, as shown in FIG. 1C). To accomplish this stimulation, two electrodes 115a, 117a, disposed apart from each other along a long axis of the forearm, are used (e.g., with one acting as the source and the other as the return of a unipolar stimulation current, or with the electrodes alternatingly acting to source or sink current for a biphasic stimulation current). Such an arrangement is common, since increased stimulation distance allows for deeper penetration of the stimulating current/voltage field (leading to increased amounts of muscle fibers stimulated), while specific muscles can be accurately targeted by selecting the appropriate pair of electrodes that are disposed, on the skin, above the targeted muscle.

The present disclosure provides embodiments of improved wearable muscular stimulation systems that are able to reliably stimulate the muscles that move the wrist and hand while comporting to the form factor of a smartwatch. These systems include a plurality of electrodes that are disposed in contact with skin at the wrist of a wearer (e.g., within a few centimeters of the head of the ulnar bone with respect to distance along a long axis of the forearm) and that are located at respective different locations encircling the wrist, thereby allowing currents to be selectively injected into/sunk from a range of locations around the wrist. This significantly simplifies the process of donning, calibrating, using, and doffing such a system while still allowing a large number of different sets of muscle fibers (and corresponding wrist/hand/finger motions) to be reliably stimulated. Additionally, by incorporating such an electrical stimulation system into the smartwatch form factor, the use of such a system can be more easily incorporated into a user's ‘regular’ usage patterns (e.g., for users who already regularly wear a smartwatch, or who would be enticed into doing so by the combination of the functionality of the electrical stimulator and of the smartwatch). Further, such a ‘common’ wearable form factor can also improve the social acceptability and/or cosmesis of the stimulation system, further improving usability.

FIG. 1B depicts an example of such a wrist-mounted stimulator 110b, with a ring of electrodes in contact with skin of the wrist of the arm 101 in respective locations circumferentially around the wrist (the stimulator 110b also includes a display and is configured to provide various functionalities of depicted is a smartwatch). FIG. 1D depicts the use of such a system to stimulate a particular muscle (flexor indicis longus, as shown in FIG. 1D), and thereby cause motion of the wrist and/or hand (flexion of the index finger, as shown in FIG. 1D). To accomplish this stimulation, two electrodes 115b, 117b, disposed on opposite sides of the wrist, are used (e.g., with one acting as the source and the other as the return of a unipolar stimulation current, or with the electrodes alternatingly acting to source or sink current for a biphasic stimulation current).

It is not obvious that such an arrangement should be usable to reliably and specifically stimulate multiple different muscles of the wrist or hand, since the muscles of the wrist and hand, at the location of the wrist, are relatively narrow, nearby each other, and may be very deep relative to the skin at the wrist. However, as demonstrated by the experimental results below, a system as described herein can be operated to provide such stimulation of targeted muscle reliably, comfortably, and efficiently. This is accomplished for a given targeted muscle by applying stimulation through a corresponding one of the electrodes while routing return current via two or more return electrodes on the opposite side of the wrist. Passing currents between electrodes on opposite sides of the wrist allows the current/voltage field to penetrate deeply, reaching deeper muscle fibers. The use of more electrodes on the side opposite the targeted muscle (e.g., a single electrode on the targeted side, and seven return electrodes on the opposite side) allows the current/voltage field to be more spread out on the non-targeted side, allowing the targeted muscle to be stimulated by the single (or otherwise fewer in number) electrode on the targeted side without stimulating any non-targeted muscles beneath any of the return electrodes.

FIG. 2 illustrates, by way of example, the arrangement of a set of electrodes 200 (including individual electrodes 201a-l) circumferentially around a wrist of an arm 201. Also illustrated are the locations, in cross-sectional view, of the muscles of the wrist and hand as they pass through the wrist at the location of the electrodes, including flexor digitorum superficialis 203a (which can flex the finger(s)), extensor pollicis longus 203b (which can extend the thumb), extensor indicis 203c (which can extend the index finger), and flexor carpi ulnaris 203d (which can flex the wrist). As noted above, specific subsets of the set of electrodes 200 could be used as stimulation and return electrodes, with the stimulation and return electrodes located on opposite sides of the wrist, and the return electrodes outnumbering the single (or more) stimulation electrode, and the identity of the stimulation electrode selected based on the muscle(s) to be stimulated.

So, in the example illustrated in FIG. 2, the flexor carpi ulnaris 203d can be stimulated (e.g., to flex the wrist and/or to provide haptic feedback to a user in the form of such flexion and/or the activation of the flexor carpi ulnaris 203d itself) by using, as a single stimulation electrode, a first electrode 201k located on the skin proximate to the flexor carpi ulnaris 203d. A subset of the electrodes opposite the first electrode 201k, which includes electrodes 201b-h, are used as return electrodes, to allow the current injected into/sunk from the first electrode 201k to be balanced without stimulating other muscles in the wrist (e.g. muscles proximate to any of the subset of the electrodes 200 used as return electrodes). The two electrodes on each side of the first electrode 201k (201a, 201l, 201j, and 201i) are not used to stimulate or as return electrodes and may instead be set to a “high impedance” mode, so as to minimize the amount of leakage or other unwanted current that might pass through those electrodes. These flanking electrodes may be set to high-impedance or otherwise not used for stimulation or return of stimulation current in order to, e.g., cause the current through the first electrode 201k to penetrate more deeply into the wrist, thereby improving the stimulation of the target muscle 203d.

As shown in FIG. 2, a system as described herein may include twelve electrodes and, to stimulate a target muscle, could select a corresponding single electrode to act as the stimulating electrode and use the set of seven electrodes opposite the stimulating electrode as return electrodes, with two electrodes on either side of the stimulating electrode used as neither stimulating nor return electrodes (e.g., operated in a high-impedance mode). However, this is only one possible example of a configuration or operation of a system as described herein. For example, such a system could have more or fewer than twelve electrodes. Additionally or alternatively, the pattern of stimulating and return electrodes could differ. For example, more or fewer than two electrodes flanking the stimulating electrodes could be used as neither stimulation nor return electrodes (e.g., a set of nine opposite electrodes could be used as return, or five or three). Indeed, the pattern of stimulating and return electrodes could be non-symmetric, e.g., as a result of a more extensive calibration process determining specific, and not necessarily symmetric, patterns of stimulating and return electrodes for each targeted muscle.

In order to stimulate a specified target muscle, a particular stimulating electrode, of the set of electrodes encircling the wrist, can be identified and used to provide stimulation current (with a corresponding set of, e.g., seven electrodes on the opposite side of the wrist being used as return electrodes). Identifying the particular stimulating electrode for the specified target muscle can include using a lookup table or similar mapping or other calibration information to determine which of the electrodes is to be used for the specified target muscle. Additionally or alternatively, relative motion between the set of electrodes on the surface of the wrist and the underlying musculature (e.g., as a result of a user rotating their wrist) could be detected and used to determine which electrode to use to stimulate the specified target muscle. For example, a ‘default’ electrode for a muscle could be determined and then, based on a detected rotation of the wrist, the stimulation electrode selected as a shift from the ‘default’ by an amount and direction related to the detected rotation. In another example, separate calibration data could be determined for each of a set of rotations or postures of the wrist/hand, with respective mappings between target muscles and the electrodes to use for stimulation thereof. In such an example, the rotation of the wrist can be detected and used to determine which set of calibration data to use to determine which electrode to use to stimulate the target muscle. The rotation of the wrist can be detected in a variety of ways, e.g., using an accelerometer, gyroscope, magnetometer, inertial measurement unit, and/or some other sensor(s).

Constraining the positioning and number of electrodes to a band that encircles the wrist at a single location constrains the space of post-application calibration as well as reducing the effort and increasing the convenience of donning/doffing the set of electrodes. For example, a device as described herein could be calibrated, after mounting to/around the wrist of a wearer, by stepping through each electrode in turn, gradually increasing the current delivered therethrough (with a set of opposite electrodes acting as returns) until muscle stimulation is reliably achieved (or until a maximum stimulation level is reached). The level of stimulation that resulted in reliable stimulation, along with the identity of the muscle stimulated/movement induced by the stimulation can then be recorded (e.g., in a memory of a simulator device) as calibration data. Such a process is significantly reduced relative to the calibration process for, e.g., a conventional forearm-based muscle stimulation system, since the locations of each individual electrode may be adjusted multiple times in order to improve recruitment of one or more target muscles, after which stimulation may be ramped for a large number of pairwise combinations of stimulation and return electrode in order to fully calibrate such a system.

Additionally, by constraining the electrodes to a regular arrangement around the wrist, the mounting and/or calibration process for subsequent uses is simplified to ensuring that the device is mounted at the correct angle relative to the wrist and location along the long axis of the forearm, a significant simplification to upper-arm systems wherein the location of individual electrodes may require adjustment and/or an electrode-containing garment may require re-alignment with the skin at many different locations. Indeed, in some examples a stimulator device as described herein could operate to detect a difference in the angle of alignment of the electrodes of the system relative to when a set of calibration information was generated and, based on the detected difference, use a correspondingly shifted version of the calibration data or otherwise adapt to the detected difference in angle.

As demonstrated by the experimental results below, a system as described herein can operate to reliably stimulate specific muscles of the wrist and hand using a ring of electrodes encircling the wrist. This stimulation can be achieved efficiently, using less than 15 milliamps of current injected through a stimulating electrode (and returned via two or more return electrodes on the opposite side of the wrist). Such stimulation can be achieved using a driving voltage of less than 100 volts. When using a set of twelve such electrodes, six or more muscles can be reliably and selectively stimulated using such a system across a variety of wrist angles/postures. If the population of target muscles is restricted, reliable stimulation can be achieved using fewer electrodes. Similarly, the use of more than twelve electrodes, approximately equally spaced around the wrist, can reliably stimulate more than six target muscles.

These benefits can be obtained by a set of electrodes that, when mounted to the wrist at the appropriate location along the long axis of the forearm, contact respective areas of skin of the wrist that span sufficient distances along the long axis of the forearm that the target muscles can be reliably stimulated. For example, the electrodes could span at least 2 centimeters along the long axis of the forearm. In order to allow the mounting location to deviate more significantly from the appropriate location and/or to allow more muscles to be reliably stimulated, the electrodes could span a larger distance along the long axis of the forearm. For example, the electrodes could span at least 4 centimeters along the long axis of the forearm to allow a system with twelve such electrodes to reliably stimulate eight different muscles across a range of wrist angles/postures. Additionally or alternatively, such a system could include multiple different rings of such electrodes, at respective different locations along the long axis of the forearm, to compensate for misalignment of the device (e.g., by selecting the ring of electrodes that corresponds to the appropriate location along the long axis of the forearm), to allow more muscles to be stimulated (e.g., some muscles could be stimulate-able using a ring of electrodes located closer to the end of the ulnar bone, while other muscles could be stimulate-able using a ring of electrodes located farther away), or to provide some other benefits.

A wrist-mounted muscle stimulation system as described herein can be used to facilitate a variety of applications. For example, muscle stimulation could be provided as part of a haptic interface to indicate information to a user. E.g., to stimulate muscles of the wrist and hand to indicate navigation directions, to indicate whether a person is home, to provide force feedback as part of a game, simulation, or other program, or to provide some other information haptically. Such haptic feedback could be used as part of a training or guidance application, e.g., to indicate the correct timing of a beat to a person learning how to drum or to indicate the correct timing of some other action.

A wrist-mounted muscle stimulation system as described herein can be used as part of a medical treatment or rehabilitation. For example, to assist a person who has experienced stroke, spinal cord injury, nerve damage, or some other injury in re-learning to use the muscles of their wrist and hand by providing an initial stimulation to activate the target muscle, followed by the person attempting to activate the target muscle on their own. In another example, a person's low-magnitude activation of a muscle could be detected and, in response, the muscle electrically stimulated to enhance the person's activation thereof and/or to facilitate rehabilitation. In yet another application, a tremor or other unwanted muscle activation could be detected and cancelled out by counter-stimulation (of an opposing muscle, or of the muscle itself). In some applications, a system as described herein could be used as part of a prosthetic device to restore activity to paralyzed muscles, e.g., to restore the ability to flex the fingers/wrist and/or to increase the stability of the wrist by stimulating wrist/finger flexors to balance voluntary activation of wrist/finger extensors.

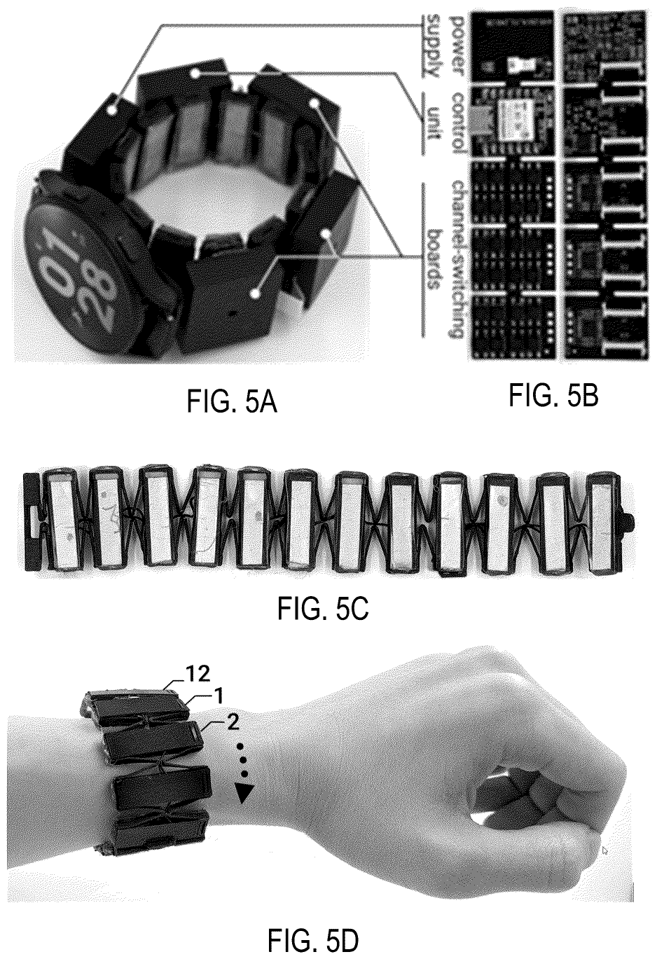

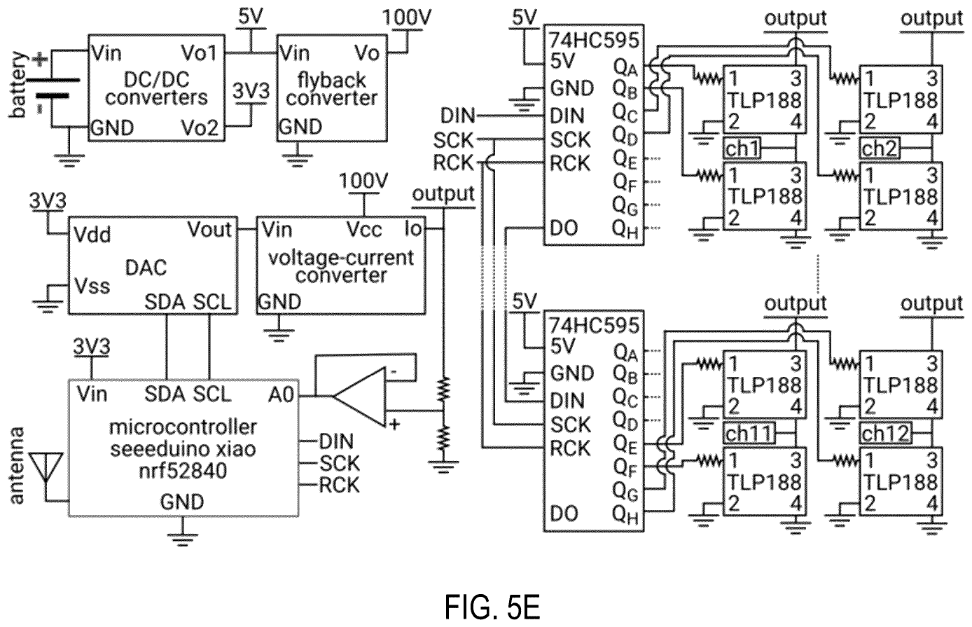

The band of a system as described herein could be configured in a variety of ways. For example, the band could include a set of rigid and/or flexible linkages (e.g., as in FIG. 5C/5D) to maintain a relatively even spacing between the electrodes around the circumference of the wrist. In some examples, a wrist-mounted device as described herein could include a display or other user interface elements. Such elements could be used to calibrate the system (e.g., to provide user indications of the sufficiency of provided stimulation and the identity of the muscle stimulated/movement induced) or to provide other functionality, e.g., smartwatch functionality. Additionally or alternatively, a wrist-mounted device as described herein could include wired or wireless communications functionality to allow such calibration or other operations to be performed via the user interface of a smartwatch, smartphone, laptop, or other system or systems. In some examples, a wrist-mounted device as described herein could include mounting features to mount a body of a watch (e.g., of a smartwatch in wireless communication with the system) to the system. For example, the system could include pins, rods, pits, holes, grommets, or other features configured to couple, as a replacement band, to the body of a smartwatch or other watch body. Such functionality could allow a system as described herein to be seamlessly integrated into a user's normal use of a smartwatch.

### II. Example Systems

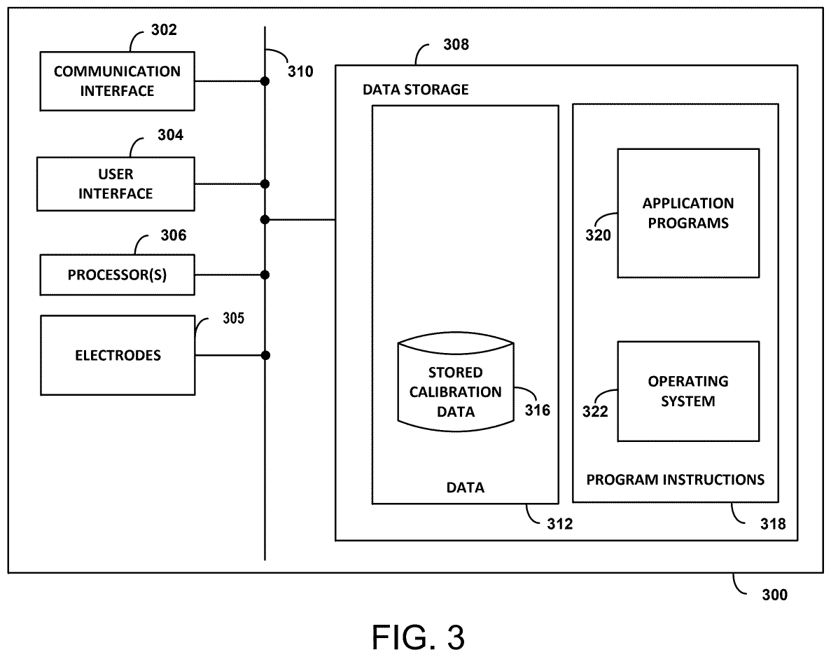

FIG. 3 illustrates an example system 300 that may be used to implement the methods and/or apparatus described herein. By way of example and without limitation, system 300 may be or include a computer (such as a desktop, notebook, tablet, or handheld computer, a server), elements of a wearable system (e.g., a system configured to be worn on the wrist of a wearer), elements of an assistive device, or some other type of device or system or combination of devices and/or systems. It should be understood that elements of system 300 may represent a physical instrument and/or computing device such as a server, a particular physical hardware platform on which applications operate in software, or other combinations of hardware and software that are configured to carry out functions as described herein.

As shown in FIG. 3, system 300 may include a communication interface 302, a set of electrodes 305 operable to deliver stimulation to a wrist (e.g., stimulation electrodes configured to be applied at regular locations around the wrist, e.g., by incorporation into a flexible or non-flexible band), a user interface 304, one or more processors 306, and data storage 308, all of which may be communicatively linked together by a system bus, network, or other connection mechanism 310.

Communication interface 302 may function to allow system 300 to communicate, using analog or digital modulation of electric, magnetic, electromagnetic, optical, or other signals, with other devices (e.g., with systems providing sets of haptic outputs to be delivered to a wearer of the system 300 via stimulation of muscle fibers, sets of muscle stimulation outputs to be implemented in muscles of the wrist of the wearer to facilitate a rehabilitative and/or prosthetic application), access networks, and/or transport networks. Thus, communication interface 302 may facilitate circuit-switched and/or packet-switched communication, such as Internet protocol (IP) or other packetized communication. For instance, communication interface 302 may include a chipset and antenna arranged for wireless communication with a radio access network or an access point. Also, communication interface 302 may take the form of or include a wireline interface, such as an Ethernet, Universal Serial Bus (USB), or High-Definition Multimedia Interface (HDMI) port. Communication interface 302 may also take the form of or include a wireless interface, such as a WiFi, BLUETOOTH®, global positioning system (GPS), or wide-area wireless interface (e.g., WiMAX, 3GPP Long-Term Evolution (LTE), or 3GPP 5G). However, other forms of physical layer interfaces and other types of standard or proprietary communication protocols may be used over communication interface 302. Furthermore, communication interface 302 may comprise multiple physical communication interfaces (e.g., a WiFi interface, a BLUETOOTH® interface, and a wide-area wireless interface).

User interface 304 may function to allow system 300 to interact with a user, for example to receive input from and/or to provide output to the user. Thus, user interface 304 may include input components such as a keypad, keyboard, touch-sensitive or presence-sensitive panel, computer mouse, trackball, joystick, microphone, and so on. User interface 304 may also include one or more output components such as a display screen which, for example, may be combined with a presence-sensitive panel. The display screen may be based on CRT, LCD, and/or LED technologies, or other technologies now known or later developed. User interface 304 may also be configured to generate audible output(s), via a speaker, speaker jack, audio output port, audio output device, earphones, and/or other similar devices. The user interface 304 may be operable to permit a user to initiate a calibration procedure and to provide feedback related thereto (e.g., to indicate whether stimulation provided by the system reliably provoked a perceptible muscular stimulation and/or to indicate which muscle was stimulated and/or which type of hand or wrist motion resulted from the stimulation), allowing stimulation magnitude and pattern calibration data to be generated and/or input, or to perform some other operation. Additionally or alternatively, such calibration data can be obtained via the system 300 communicating, using the communication interface 302, with other systems that include a user interface, e.g., with a smartphone and/or with a watch (e.g., a watch body) that is coupled to mounting features on the system 300.

Processor(s) 306 may comprise one or more general purpose processors-e.g., microprocessors-and/or one or more special purpose processors-e.g., digital signal processors (DSPs), graphics processing units (GPUs), floating point units (FPUs), network processors, tensor processing units (TPUs), or application-specific integrated circuits (ASICs). Data storage 308 may include one or more volatile and/or non-volatile storage components, such as magnetic, optical, flash, or organic storage, and may be integrated in whole or in part with processor(s) 306 and/or with some other element of the system. Data storage 308 may include removable and/or non-removable components.

Processor(s) 306 may be capable of executing program instructions 318 (e.g., compiled or non-compiled program logic and/or machine code) stored in data storage 308 to carry out the various functions described herein. Therefore, data storage 308 may include a non-transitory computer-readable medium, having stored thereon program instructions that, upon execution by system 300, cause system 300 to carry out any of the methods, processes, or functions disclosed in this specification and/or the accompanying drawings. The execution of program instructions 318 by processor(s) 306 may result in processor 306 using data 312.

By way of example, program instructions 318 may include an operating system 322 (e.g., an operating system kernel, device driver(s), and/or other modules) and one or more application programs 320 (e.g., functions for executing the methods described herein) installed on system 300. Data 312 may include stored calibration data 316 (e.g., stored sets of stimulation thresholds and muscle/movement identities to induce muscle stimulation/movements of the fingers and/or wrist) that can be used to determine how to operate the electrode(s) 305 to stimulate muscle fibers of a user.

Application programs 320 may communicate with operating system 322 through one or more application programming interfaces (APIs). These APIs may facilitate, for instance, application programs 320 transmitting or receiving information via communication interface 302, receiving and/or displaying information on user interface 304, and so on.

Application programs 320 may take the form of “apps” that could be downloadable to system 300 through one or more online application stores or application markets (via, e.g., the communication interface 302). However, application programs can also be installed on system 300 in other ways, such as via a web browser or through a physical interface (e.g., a USB port) of the system 300.

The electrodes 305 may be driven by one or more stimulators, which may include high voltage generators, amplifiers, switches, controlled-current and/or controlled-voltage sources, clocks, current and/or voltage-limiting elements, or other elements to controllably generate currents, voltages or other energies that can be delivered to a user's wrist via the electrodes 305. In some examples, the stimulator(s) can be configured to generate a single controlled current/voltage at a time, and to operate an array of switches to deliver that single controlled current/voltage to a specified stimulation electrode and return electrode(s) of the electrodes 305, with different levels of stimulation provided via multiple different stimulation electrodes by operating the array of switches in a time-division multiplexed manner. Additionally or alternatively, the stimulator(s) could include multiple stimulator systems capable of generating respective controlled currents/voltages at a time.

### III. Example Methods

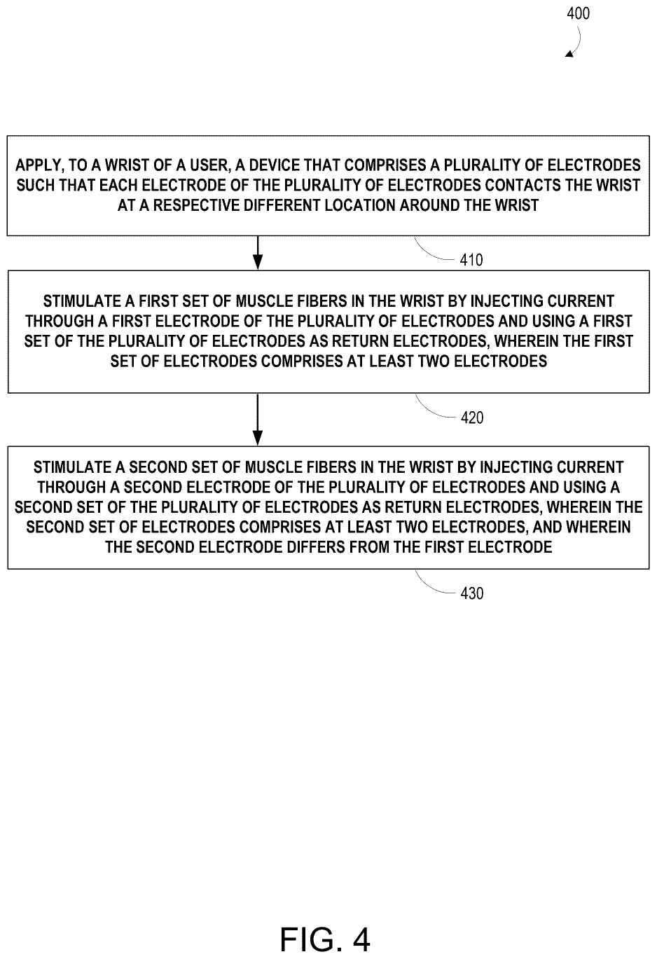

FIG. 4 depicts an example method 400. The method 400 includes applying, to a wrist of a user, a device that comprises a plurality of electrodes such that each electrode of the plurality of electrodes contacts the wrist at a respective different location around the wrist (410). The method 400 additionally includes stimulating a first set of muscle fibers in the wrist by injecting current through a first electrode of the plurality of electrodes and using a first set of the plurality of electrodes as return electrodes, wherein the first set of electrodes comprises at least two electrodes (420). The method 400 yet further includes stimulating a second set of muscle fibers in the wrist by injecting current through a second electrode of the plurality of electrodes and using a second set of the plurality of electrodes as return electrodes, wherein the second set of electrodes comprises at least two electrodes, and wherein the second electrode differs from the first electrode (430). The method 400 could include additional steps or features.

It should be understood that arrangements described herein are for purposes of example only. As such, those skilled in the art will appreciate that other arrangements and other elements (e.g. machines, interfaces, operations, orders, and groupings of operations, etc.) can be used instead of or in addition to the illustrated elements or arrangements.

### IV. Experimental Results

As an example of the embodiments described here, an experimental system was constructed and experimentally evaluated.

Smartwatches have come into common use, making them into today's de-facto wearables. Despite advancements in sensing, haptics on smartwatches are still generally restricted to tactile feedback (e.g., vibration). Most smartwatch-sized actuators cannot render strong force-feedback. Prior attempts at electrical muscle stimulation (EMS) have provided compact force-feedback but, to actuate fingers, required users to wear many electrodes on their forearms, including upper forearms. While such forearm electrodes provide good accuracy for targeting specific muscles, they are more of an imposition to don, doff, and wear, preventing prior EMS systems from being used as a practical force-feedback interface.

The embodiments described herein overcome the limitations of prior attempts by, among other things, moving the electrodes to the wrist—allowing them to be conveniently packaged in a smartwatch band. Experimental evaluation to cross-sectionally stimulate the wrist in 1,728 trials showed that thumb extension, index extension & flexion, middle flexion, pinky flexion, and wrist flexion can be actuated using the embodiments described herein. A compact EMS system that integrates directly into a smartwatch's wristband (with a custom stimulator, electrodes, demultiplexers, and communication) was also developed and experimentally evaluated. Using such a system, participants were able to calibrate the device by themselves ~50% faster than with conventional EMS systems. All participants preferred the experience of the device, especially for its social acceptability & practicality relative to conventional EMS systems.

EMS allows force-feedback to be miniaturized (e.g., relative to hydraulic, pneumatic, electromagnetic, or other force-feedback actuators) since its electrodes and battery are typically smaller than the components needed for mechanical actuators. Unfortunately, prior EMS systems to actuate the fingers or the wrist placed the electrodes on the forearm or back-of-hand. Such a configuration limited the practicality of EMS—most users do not typically wear electrodes in their forearm day-to-day.

Instead, the embodiments describe herein implement EMS using electrodes located where users today would wear a smartwatch—around the wrist (see, e.g., FIGS. 1A and 1B). An actuation strategy using electrodes placed only around the wrist was experimentally evaluated, using the electrodes to stimulate cross-sectionally (e.g., forming a stimulating pair of electrodes from diametrically opposing electrodes). This enabled a wider range of finger actuation than the side-by-side electrode placement typical of prior EMS systems. Possible finger & wrist actuations when electrodes are at the wrist (including at different distances to the hand and during different hand poses) were also evaluated. It was confirmed that the systems and methods described herein can render thumb extension, index extension & flexion, middle flexion, pinky flexion, and wrist flexion—all using only electrodes disposed around the wrist. A complete “wrist-EMS device” was designed and fabricated that included a compact muscle stimulator integrates directly into a smartwatch's wristband, with a custom EMS stimulator, 12 electrodes, demultiplexers, battery, and wireless communication.

A study was conducted using these experimental devices wherein participants were asked to calibrate the EMS by themselves (with no experimenter assistance) and asked to wear the wrist-EMS in public. Participants were able to calibrate the wrist-EMS device by themselves ~50% faster than with a conventional forearm-EMS system. All participants favored the experience of using the wrist-EMS device, especially for its social acceptability & practicality (this study involved public interactions, such as at a café).

The embodiments described herein facilitate completely new everyday interactions with smartwatches, such as actuating one's hand to display walking directions, wrist-based drumming assistance, and haptic exercise coaching.

Alternative methods for generating the magnitude of forces needed to physically move a user's wrist or fingers can include bulky mechanical actuators operated as force-feedback devices (e.g., motors, exoskeletons, robotic arms, pneumatics). However, these conventional methods for achieving force-feedback do not scale down gracefully: if one scales down the mechanical actuators, the output force is greatly compromised, eventually becoming too weak to move fingers/wrists; conversely, the mechanical actuators are not scaled down, they will not fit inside the typical form-factor of a wrist-worn device.

Prior attempts at EMS have been used to actuate wrist and fingers in interactive contexts, including haptics for immersive experiences and serving as a novel information output modality. However, these prior EMS systems were inconvenient to don, doff, wear, and calibrate. In order to reliably actuate a muscle using such systems, it is necessary to: (1) place electrodes on a skin area of interest to target a muscle, likely on the upper forearm, which requires knowledge of musculature under the skin; (2) send electrical impulses via the stimulator, gradually increasing the intensity and observing the result—a muscle contraction and target limb's movement; and (3) if the contraction is not achieved or not sufficiently robust (e.g., the user might rotate their limb or body to check if this deteriorates the quality of the observed actuation), step (1) is repeated, re-placing electrodes in a new area of interest.

These steps illustrate two challenges that undermine the practicality of prior EMS systems: (1) the need for manual calibration, requiring users to tweak the stimulation parameters with each use; and (2) the need for many electrode locations, requiring users to either try stimulating many skin areas or wear sleeves that cover their skin with many electrodes, so that at least some of the electrodes contact the correct areas.

The need for prior EMS systems for many electrode locations on the forearm and/or highly accurate placement at a smaller number of locations diminishes the practicality of those systems. The use of, e.g., garments covering a large skin area with high-density distributions of electrodes, so that at least some electrodes reach the correct areas, do not blend well with everyday interactions nor with any existing devices and are more effortful and inconvenient to don/doff—in other words, these prior EMS systems are an additional device that the users must attach to their body that does not integrate directly in any existing devices they might be already wearing.

Prior EMS systems that actuate the fingers/wrist place the electrodes on the forearm or, in rare cases, the back-of-hand. Such placement corresponds to the location of the bulk of the muscle fibers (and, indeed, the motor points of those muscles) that actuate the wrist and most degrees of freedom of the fingers. Indeed, Bao et al. concluded, based on an examination of the entire skin from the wrist up to the elbow for EMS finger actuation, that it was necessary for at least one electrode to be placed on the forearm. However, the forearm is an impractical electrode location since users do not wear any other devices in this area (with which the EMS could be integrated) and since the unconstrained expanse of forearm also increases the number of possible electrode locations—making calibration, especially by non-clinal end-users, less practical. While back of the hand electrode stimulation is able to flex individual fingers, it cannot be used to extend the fingers, and, like forearm-EMS, such placement does not correspond with any devices users might already commonly use—making this form factor also very impractical for everyday use of EMS.

A novel form factor EMS device, according to the embodiments described herein, was developed that integrates electrodes into a smartwatch-form factor device, a form factor that users are already familiar with. The specific configuration of those electrodes and the pattern of their use to provide electrical stimulation allows this form factor to reliably and comfortably electrically actuate a wide variety of movements of the wrists and fingers. The embodiments described herein, which move all electrodes to the wrist, contrasts with conventional EMS approaches for actuating wrist/fingers, which place electrodes on the forearm.

The embodiments described herein are able to stimulate muscles of the hand and wrist because, while most finger/wrist muscles are largest under the forearm, some portion of many of those muscles (see., e.g., FIG. 2) are located in or proximate to the wrist. For example, the index finger extensor (extensor indicis) 203c passes close to the skin surface, around the back of the wrist. As shown experimentally herein, many muscles were accessible to the experimental EMS system, such as the thumb (extensor pollicis longus) 203b, the index finger (flexor digitorum superficialis) 203a, and the wrist (flexor carpi ulnaris) 203d.

The conventional EMS electrode placement strategy employed in the forearm (and virtually all other locations) includes placing two electrodes side by side, along the long axis of the muscle and atop the muscle, as depicted by electrodes 115a, 117a in FIG. 1C. This can also have the benefit of, e.g., allowing efferent nerves to the muscle to be recruited directly. However, it was found that this conventional electrode placement does not work when translated to the wrist unless modified to include electrodes at other locations, e.g., a ground electrode on the elbow.

In contrast, the experimental wrist-EMS device stimulates cross-sectionally through the wrist, e.g. as depicted by electrodes 115b, 117b located on opposite sides of the wrist in FIG. 1D. This was experimentally demonstrated to result in a wider range of finger actuations, achieving similar functional outcomes for some muscles and/or wrist/finger movements as forearm-EMS. This arrangement increases the electrode-to-electrode distance, creating a deeper current path inside (through) the wrist. This electrode configuration and the accompanying stimulation strategy described herein facilitates the wrist-EMS or other systems as described herein to be easily integrated into a device—the smartwatch—that many users are already familiar with.

The novel stimulation technique and form factor for EMS described herein allows EMS to be easily and reliably used in practical settings, outside the laboratory. This approach has a number of practical benefits: (1) it provides an extremely compact form factor for simple EMS actuations of some fingers and the wrist; (2) it reduces calibration time, even when users are new to EMS calibration—it reduces calibration by 50% compared to an improved version of the conventional forearm EMS; (3) it provides new avenues for social acceptability of EMS, given that no electrodes are visible on the forearm, since they are tucked under the smartwatch—none of the participants in the studies described herein felt uncomfortable when it came to public-facing interactions; and, finally (4) this approach allows a single smartwatch to perform both pose sensing and pose actuation.

The experimental device, shown in various aspects in FIGS. 1B and 5A-E, was directly integrated into the watchband of an Android smartwatch (Samsung, Galaxy Watch 5). Inside the wristband, five modular PCBs were integrated that performed signal generation, signal amplification, demultiplexing, and wireless communication. 12 electrodes were radially placed on the backside of the expandable wristband.

A PLA 3D-printed flexible watchband was fabricated that expanded between 147-360 mm in circumference and ensured electrodes were distributed evenly circumferentially around the wrist (FIG. 5C).

Each electrode measured 10 mm×30 mm, and included a 3D printed base, copper tape, and a conductive gel sheet (Yushiro Chemical Industry, wizard gel). The copper electrode is directly connected to the channel switching board (demultiplexer). The addition of conductive gel improved impedance matching, resulting in a more comfortable sensation, similar to pre-gelled electrodes.

Atop the wristband, five 240 mm×300 mm×80 mm 3D-printed boxes housed each of the modular PCBs. To distribute signals from each module, flexible flat cables were used.

FIG. 5E depicts the high-level circuit design of the system. This circuitry was configured to generate an appropriate signal to induce muscle stimulation when routed through the appropriate electrodes of the device. The experimental circuit design was capable of a maximum stimulation of 100V at 15 mA.

Three voltage sources were included: a 3.3 V supply via a DC-DC buck converter from the LiPo battery (3.7 V, 110mAh), which powered the microcontroller; a 5 V supply via a DC-DC boost converter from the LiPo; and the 100 V via a DC-DC flyback converter from the 5V. The 5 V was supplied to the channel switching demultiplexers and battery-charging circuit. The 100 V was used for the EMS.

By toggling a switch on our PCB, the circuit was able to switch between constant voltage and constant current stimulation. In constant-voltage mode pulse-width modulation was used to vary the intensity of EMS—always at 100 Vpp, but with varying duty cycles. Conversely, in constant-current mode, the output of a voltage-current converter was controlled using the digital-to-analog converter (DAC) connected to the microcontroller (0 V to 3.3 V) to regulate the current between 0-15 mA. Since medically compliant EMS devices typically use constant-current stimulation due to their improved safety, this method was used in all of the experiments.

A microcontroller (Seeeduino, nRF52840) with Bluetooth low energy (BLE) controlled functionality and communication. The microcontroller implemented a simple BLE protocol that target applications can use to request muscle stimulation. Target applications sent the EMS parameters (channel identity, intensity, pulse width, frequency) to the device.

A channel-switching circuit was implemented to allow the signal generator to be routed to any of the 12 electrodes. Using the demultiplexer, each electrode can be connected to the signal generator in one of three states: high (connected to the generator), ground (0 V), and HiZ (high-impedance mode). The stimulation current flowed from the electrodes set ‘high’ to the body and returned to the device through the ground electrode. HiZ electrodes were not connected to any source, so no current flowed through them.

Each channel-switching PCB included an 8-bit shift register (Nexperia, 74AHCT595) and eight photocouplers (Toshiba, TLP188). The shift registers'eight output pins were connected to the anodes of each photodiode. Each pair of photocouplers formed one half-bridge. Thus, each channel-switching PCB controlled four channels, and with three of these, the device was able to switch a total of 12 output channels.

The arm locations where users generally find it most suitable to wear a smartwatch were experimentally evaluated in order to inform subsequent experimentation. 11 participants were recruited (three women, eight men; 24.8±2.6 years old). Participants were asked to wear a smartwatch (Samsung, Galaxy Watch 5) on any arm and place it in the location where they usually wear this type of device—they were not instructed in any other way. After participants placed and adjusted the device to their preference, the distance from the head of the ulnar bone to the edge of the watch on the distal side (i.e., away from the body) was measured. Then, the experimenter proceeded by manually moving the device up the participant's arm (in the proximal direction, i.e., towards the body) by 5 mm at a time. At every position, the participant reported whether this position was acceptable; this was repeated until they reported a position that was no longer acceptable. Finally, this process was repeated to determine the acceptable position in the distal direction (i.e., away from the body and towards the hand).

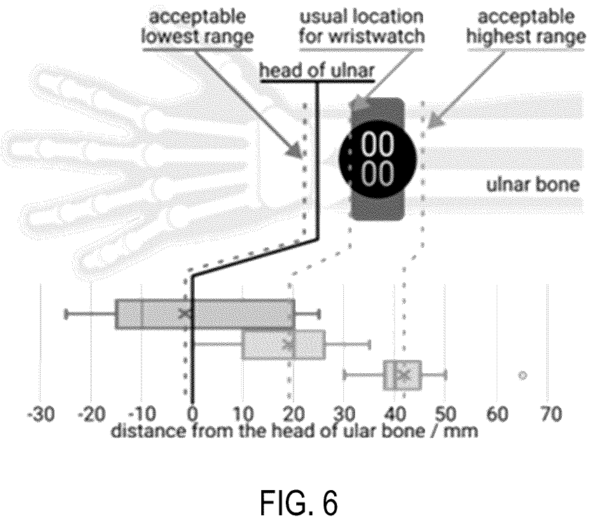

FIG. 6 depicts the experimentally measured average positions (mean±confidence interval) where participants wore smartwatches (usual & max/min). As depicted in FIG. 6, the usual preferred position was 18.9±6.7 mm (mean±confidence interval using a t distribution), with the highest position towards the body at 41.5±6.5 mm, and the lowest away from the wrist at −1.5±12.1 mm. The latter position's variation was larger since participants who wear wristwatches often described that they did not care if the device fell toward the hand.

Based on this experimentally-validated range where the smartwatches are worn (between 0 -40 mm, measured from the head of the ulnar bone), the possibility of using only electrodes at this wrist location to actuate wrist/fingers via cross-sectional electrical stimulation was assessed experimentally, along with the identity of the joints flexed or extended thereby. 12 participants were recruited (six male and six female; 24.3±4.2 years old). Nine wore their watches on their left arm and the rest on their right hand.

The prototype described above (including a 12-electrode ring-shaped band, demultiplexers, etc.), except for the signal generator, was used. Instead, for this initial experiment, a medically compliant EMS stimulator (HASOMED, Rehamove3) was used, which was capable of stimulation up to 100 mA. The results of this initial study informed the design of the system described above, since it was observed that only 15 mA of current was needed to reliably stimulate an acceptable range of muscles/movement—thus leading to a smaller circuit, without large high-current transformers. The stimulation was a sequence of pulses at 100 Hz, and each pulse-width was 200 μs in duration. The first electrode was adjusted to sit atop each participant's finger extension muscle (extensor indicis). The remainder of the electrodes landed in their nominal position as the band was wrapped around the participants'wrists.

This study was performed at three wrist locations: 20 mm, 30 mm, and 40 mm from the head of the ulnar bone—all acceptable ranges based on the prior experiment.

For each location, the device performance was evaluated under four different hand poses: palm facing up/down/left/right. This evaluation allowed the effects of the rotation of the skin independently from the muscles as the wrist rotates to be assessed.

Stimulation provided to the muscles of the wrist according to the embodiments described herein uses electrodes on opposing sides of the participants'wrists. In this study, the procedure explored all twelve electrodes, rotating between them one by one. For each selected stimulation electrode, the seven electrodes on the opposite side acted as a common ground—the remaining four (two on each side of the selected stimulation electrode) were turned off (HiZ mode) and thus neither stimulating nor ground. Extensive pilot experiments demonstrated such an arrangement to be broadly effective and to provide superior results to alternative patterns.

Per trial, the stimulation started at 0 mA and increased by 1 mA at a time until either a full contraction was achieved, or the participant reported discomfort. This was repeated for all channels at three locations (with respect to distance along the long axis of the arm) & four hand poses.

For each trial, the participant's hand was filmed via two video cameras (top and thumb's side). The videos were analyzed to extract the frames depicting the maximum pose reached per trial. All joint angles were annotated and extracted.

Each participant was stimulated for a total of 144 trials, leading to a total of 1728 trials across all participants. From these 1728 trials, angles were obtained for 22 degrees of freedom (5 fingers×4 DoF per finger+2 DoF for the wrist), resulting in 38,016 data points.

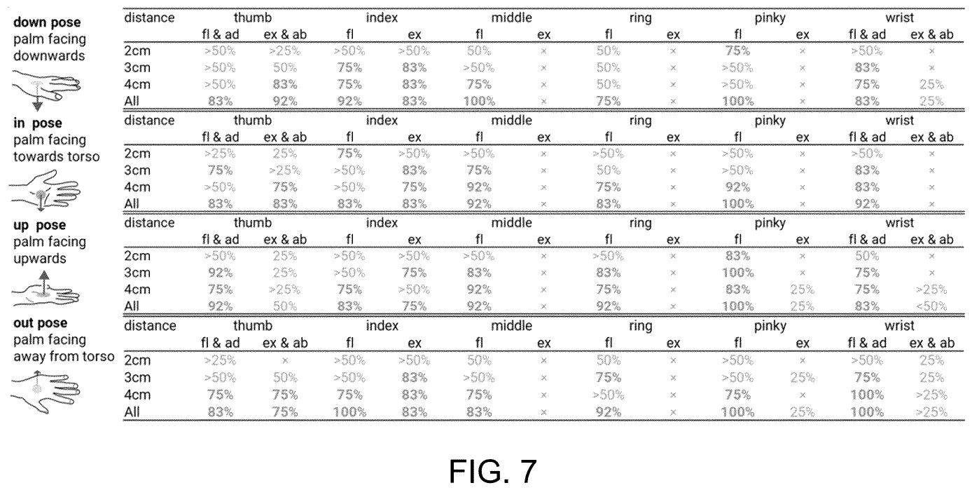

FIG. 7 depicts an overview of the experimental results, summarizing what movements are possible when the stimulation is applied at the wrist. Each entry denotes the percentage of participants for which this actuation reliably occurred with an angle larger than 5°. A reliable actuation occurred over 75% of the trials (colored green). Values 74%−25% are shown in gray. Values <25% are shown as×. Fl. stands for flexion, ex. for extension, ad. for adduction, and ab. for abduction. This same threshold for classifying an actuation as reliable was applied across hand poses (e.g., if it can be performed in 3 out of 4 hand poses). All joints (DIP: distal interphalangeal, PIP: proximal interphalangeal, and MP: metacarpal phalangeal) are combined—however, typically only PIP and MP were observed. The adduction and abduction of the four fingers, except for the thumb, are not shown because those were observed less reliably (<25%).

Results for 2 cm (no reliable actuations). Only two movements were observed at the 2 cm location (e.g., index flexion and pinky flexion), but these did not match the reliability criteria (reliable over 75% of hand poses).

Results for 3 cm (up to two actuations). With the electrodes placed 3 cm away from the wrist, two reliable actuations were observed: (1) index extension and (2) wrist flexion for all hand poses.

Results for 4 cm (up to six actuations). 4 cm was the most expressive location with respect to the number of unique actuations. This included up to six reliable actuations: (1) thumb extension, (2) index extension, (3) index flexion, (4) middle flexion, (5) pinky flexion, and (6) wrist flexion—these were observed for most poses, except the index & thumb extension which were not reliable when the palm was facing up, and the index flexion was not reliable when the palm was facing inwards.

Results for all locations (up to eight actuations). While six movements were able to be induced from a single location (4 cm), the approach described herein was able to induce two additional finger actuations if a device was configured to make use of all three locations (2, 3, or 4 cm). This composite result added the following reliable actuations: thumb flexion and ring flexion.

These results confirm that a number of different electrode form factors can be used to reliably induce a variety of different hand or wrist motions. For example, (1) one electrode band at the best location (4 cm), (2) a wider electrode band that covers all three locations (from 2 cm to 4 cm), or (3) multiple bands (or subsets of bands) of electrodes at multiple different locations (e.g., one at the 3 cm and one at the 4 cm location). While a single wider electrode band, or multiple electrode bands spanning a wider area, could reliably actuate eight movements, such a configuration would add ~1 or 2 cm in width to the device, potentially limiting wearability. The self-contained wrist-EMS experimental device was designed to operate using a single electrode band at 4 cm, in order to reliably actuate six finger/wrist movements

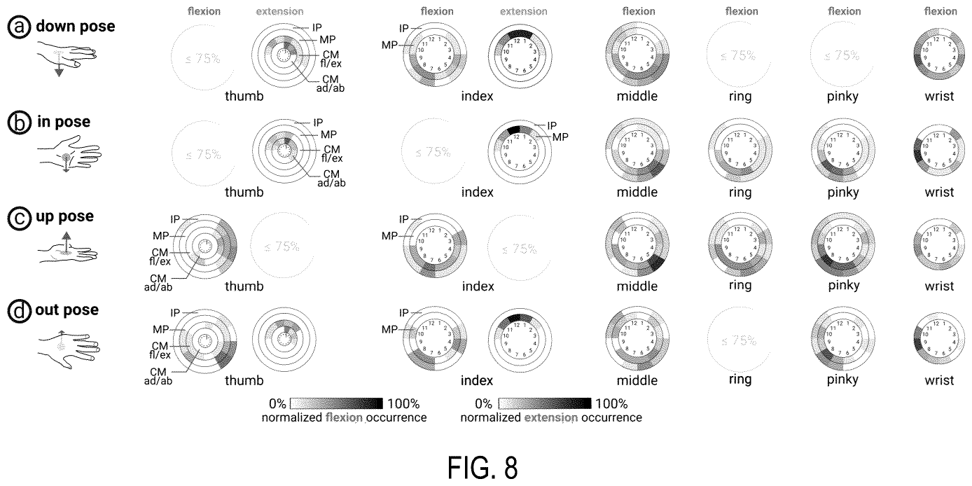

The relationship between electrode position and identity of stimulated muscle/movement was experimentally assessed, in particular analyzing the joints that successfully moved for >75% of the participants at the most expressive location (4 cm). Each joint was analyzed with respect to which electrode positions (from the 12 possible channels) were most effective in actuating this joint. FIG. 8 depicts a heatmap of which electrode channels achieved which movements, with blue shades for flexion and red shades for extension. The results show which electrode contributes to each joint flexion/extension when the palm faces (a) down, (b) up, (c) inward, and (d) outward (IP: (proximal) interphalangeal, MP: metacarpal phalangeal, CM: carpometacarpal). The darker blue and red mean that the joints were flexed and extended for more participants, respectively. The ratio was normalized by the number of participants with the joint flex/extension. Conditions that did not achieve movements for >75% of the participants are greyed out.

Four actuations were consistent in specific electrode positions across most of the participants: (1) index extension was most reliably achieved at channel 12 (and directly adjacent channels) for all hand poses beside the upwards pose; (2) wrist flexion was most reliably achieved at channel 9 for all poses; (3) middle flexion was observed to be most reliably actuated at channel 5, but only when the palm was facing upward or inward; (4) pinky flexion was observed to be most reliably actuated at channel 8 for all hand poses besides downward pose.

The thumb extension was difficult to observe when the palm was facing up, likely because, in this pose, the thumb is already passively extended. Also, the pinky and ring fingers were flexed at the MP joint (unlike the index and middle, which flexed at the PIP), which suggests that the current was also stimulating the motor nerves that innervate the lumbricals or interossei muscles—as these typically flex the MP joints.

Across all trials, the elicited movements required no more than 12 mA of current. This allowed the design a more compact final circuit that was limited with respect to current to up to 15 mA, rather than of large transformers (e.g., as used in Rehamove 3), which allow for much larger currents (e.g., up to 100 mA).

Despite moving all electrodes to the wrist (a location not previously known for its accuracy concerning actuation of fingers/wrist via EMS), reliable finger/wrist actuations were observed for up to six different muscles/movements from a single location. This variety of movements can facilitate a wide range of applications using a wrist-worn device as described herein. One of the benefits of moving all electrodes to the wrist is practicality and ease of use.

Eight participants (four women and four men; 23.3±3.5 years old) were recruited for a study to assess the practical use of the experimental system; none had partaken in the above experiment. All participants wore their watches on their left arm. participants experienced two EMS conditions: forearm-EMS and the wrist-EMS device. The forearm-EMS had two electrode bands instead of one to replicate electrode placement in conventional EMS.

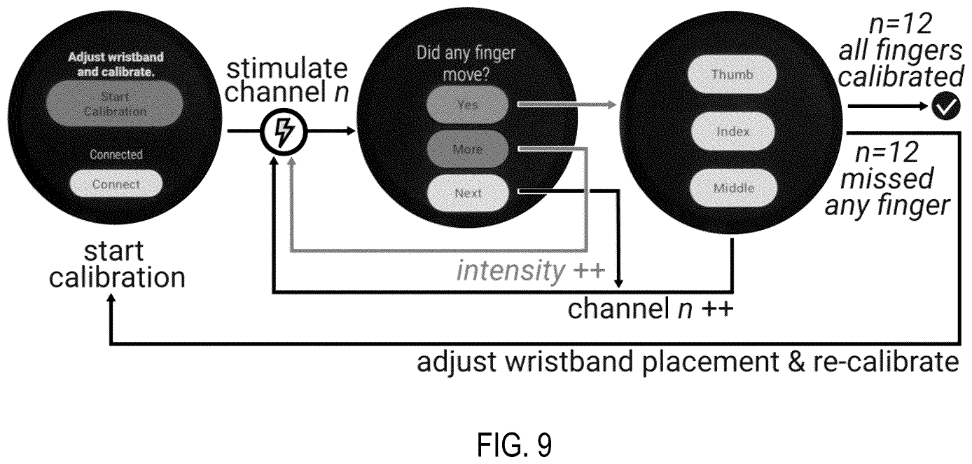

Other than the one-to-two band difference in these conditions, the remainder of the experimental setup was the same, including the stimulation hardware, the design of the electrode bands, and the calibration interface. Participants interacted via a calibration app on a smartwatch's screen, which laid flat on a table for consistency across the two interface conditions. This calibration screen allowed participants to test all 12 channels while adjusting the stimulation intensity. Aspects of the calibration interface and calibration method are depicted in FIG. 9. Once all 12 channels had been calibrated, the app automatically prompted participants to adjust the placement of the bands.

Task: calibrate the EMS by yourself. Participants were asked to wear one of the EMS interfaces (forearm or wrist) at a time and to calibrate it to “create three reliable finger movements (e.g., thumb extension, index extension; and middle flexion)”. The goal of this study was to observe how participants would calibrate without assistance from the experimenter, unlike most EMS research where experimenters calibrate each participant—another key reason why conventional EMS systems are limited to lab settings. As such, participants had to perform every aspect of the calibration by themselves, including deciding where to place the electrode bands, putting bands on/off, and adjusting the stimulation.

The two conditions were balanced. Despite the difference in the number of electrode bands, both conditions had the same 12 stimulation channels. Condition order was counter-balanced across participants.

After the wrist-EMS condition, the participants'calibrations were validated by having them use a navigation application (described in greater detail below), which was adjusted to work indoors for this experiment. Instead of walking outdoors, participants sat in a chair while the app autonomously updated their position to move along a predefined route. The wrist-EMS actuated their fingers corresponding to the turns (index finger extension for the left turn and middle finger flexion for the right), and upon arrival at the destination (thumb extension to represent a ‘thumbs up’).

Relocating EMS electrodes to the wrist and housing the hardware in a smartwatch enabled new EMS interactions, as well as facilitating and easing the use of existing EMS-based interactions outside of the lab. To evaluate this, two novel interactive applications were developed for EMS, and three existing EMS applications were extended beyond tethered laboratory settings. All the applications were implemented via Android Studio and run on a Galaxy Watch 5; all applications communicate with the EMS device via BLE.

The exceptional convenience and wearability of the wrist-based EMS systems and methods described herein (e.g., wrist-EMS) enables force-feedback in situations where conventional EMS devices are impractical due to their interference with a user's movements, are unsuitable for public use (e.g., due to unacceptably negative cosmetic effects), or their need for lengthy calibration.
Application #1: Haptic notifications for exercise. While vibrations are the most common haptic modality used for wearable notifications, the experimental device allowed users to benefit from more expressive haptics on their smartwatches. Force-feedback notifications were used during a HIIT (high intensity interval training) workout: (a) the wrist-EMS lifted up the thumb for each squat to indicate the cadence; (b) at the end of each HIIT interval, the wrist-EMS flexed the user's finger to indicate an interval is now done, and the rest will start; and finally (c) when the rest period ended, the wrist-EMS flexed either the user's index, middle, or pinky fingers to indicate which exercise is next (“1”, “2” or “3”, from a list of different exercises the user loaded for the session).
Application #2: Eyes-free GPS navigation. A user started a wayfinding application to navigate to their destination using their smartwatch but continued eyes-free. In this example, instead of looking at the screen to check turns, the EMS integrated in their smartwatch directly actuated their (a) index finger, or (b) middle finger to indicate upcoming turns. Then, (c) actuated their thumb in a “thumbs-up” gesture to indicate the arrival at the destination. This functionality was implemented via Mapbox API.

With the expanded functionality and practicality of the wrist-EMS system described herein, several existing EMS applications can now be deployed outside of lab settings.
Application #3: Haptic assistance in drumming. The compact form factor of the experimental device allowed it to be useful in a number of everyday situations, where donning an EMS device and calibrating it on the forearm would be overly laborious and/or inconvenient. In this application, a user benefitted from force-feedback while using a drum assistant application: (a) after configuring the desired beat pattern, they (b) started the EMS, which in turn actuated the wrist to render the pattern, enabling the user to follow along in time.
Application #4: Smartwatch as VR force-feedback. The most popular usage of EMS in HCl has been adding force feedback for VR; however, existing systems to achieve this require electrodes to be worn or placed in the forearm, reducing practicality for users that prefer to quickly start their VR applications. The experimental smartwatch with integrated EMS described herein doubled as a force-feedback device for a user engaged in a VR driving simulation, holding the virtual steering wheel of their F1 car. When they pressed the ignition button, they felt a resistance rendered by the device's index extension. Similarly, as they shifted gears, they felt the resistance of pushing the paddle up or down, which the device rendered by flexing the middle finger or extending the index finger.
Application #5: Actuating everyday objects with EMS. The device described herein was used, instead of the traditional forearm-EMS, to implement aspects of a smart door notification and access system. This smart door applicated provided a user who approached it with haptic information regarding whether the occupant was busy or unavailable. As the user approached the smart door, the application (running in the user's smartwatch) communicated with the door's microcontroller via BLE. When both devices were in range, the door informed the smartwatch of the occupant's status. If the occupant had set the room to “unavailable,” the smartwatch application responded by actuating the user's wrist with a “repel” gesture (achieved using EMS). Conversely, if the room was set to “busy”, the device responded with a “knock-knock” gesture (rendered by alternating EMS flexions and extensions).

### V. Conclusion

It should be understood that arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

1. A wearable system, comprising:
a plurality of electrodes;
a band, wherein the band is configured to maintain the plurality of electrodes in contact with skin of a wrist of a wearer such that each electrode of the plurality of electrodes contacts the wrist at a respective different location around the wrist; and
a stimulator, wherein the stimulator is configured to:
stimulate a first set of muscle fibers in the wrist by injecting current through a first electrode of the plurality of electrodes and using a first set of the plurality of electrodes as return electrodes, wherein the first set of electrodes comprises at least two electrodes, and
stimulate a second set of muscle fibers in the wrist by injecting current through a second electrode of the plurality of electrodes and using a second set of the plurality of electrodes as return electrodes, wherein the second set of electrodes comprises at least two electrodes, and wherein the second electrode differs from the first electrode.

2. The system of claim 1, wherein the first electrode is separated from any electrode of the first set of electrodes by at least one other electrode of the plurality of electrodes.

3. The system of claim 2, wherein the first set of electrodes are a contiguous set of the plurality of electrodes and wherein an equal number of electrodes, of the plurality of electrodes and not part of the first set of electrodes, are disposed on either side of the first electrode between the first electrode and the first set of electrodes.

4. The system of claim 3, wherein the plurality of electrodes comprises twelve electrodes, wherein the first set of electrodes comprises seven electrodes, and wherein two electrodes are disposed on each side of the first electrode between the first electrode and the first set of electrodes.

5. The system of claim 1, wherein the band is configured to maintain the respective locations of each electrode of the plurality of electrodes such that distances between neighboring electrodes of the plurality of electrodes differ by less than about 20%.

6. The system of claim 1, wherein stimulating the first set of muscle fibers in the wrist comprises injecting a current less than 15 milliamps.

7. The system of claim 1, wherein the stimulator is configured to use the plurality of electrodes to stimulate flexor digitorum superficialis, extensor pollicis longus, extensor indicis, and flexor carpi ulnaris.

8. (canceled)

9. (canceled)

10. The system of claim 1, wherein at least one of the band or the plurality of electrodes includes mounting features to mount a body of a watch to the system.

11. The system of claim 10, wherein the stimulator is configured to receive, via wireless communication from the watch, a command to provide a first stimulus, and wherein the stimulator is configured to stimulate the first set of muscle fibers in the wrist in response to receiving the command.

12. The system of claim 1, wherein at least one electrode of the plurality of electrodes, when maintained in contact with the wrist by the band, contacts an area of the wrist that spans at least 2 centimeters in a direction along a long axis of a forearm of the wearer.

13. The system of claim 1, wherein the plurality of electrodes is a first plurality of electrodes, and further comprising a second plurality of electrodes, wherein the first plurality of electrodes and second plurality of electrodes are configured to contact the wrist at respective locations that differ with respect to distance along a long axis of a forearm of the wearer.

14. A method, comprising:
applying, to a wrist of a user, a device that comprises a plurality of electrodes such that each electrode of the plurality of electrodes contacts the wrist at a respective different location around the wrist;
stimulating a first set of muscle fibers in the wrist by injecting current through a first electrode of the plurality of electrodes and using a first set of the plurality of electrodes as return electrodes, wherein the first set of electrodes comprises at least two electrodes; and
stimulating a second set of muscle fibers in the wrist by injecting current through a second electrode of the plurality of electrodes and using a second set of the plurality of electrodes as return electrodes, wherein the second set of electrodes comprises at least two electrodes, and wherein the second electrode differs from the first electrode.

15. The method of claim 14 further comprising:
injecting a plurality of different magnitudes of current during a plurality of respective different periods of time through the first electrode and using the first set of the plurality of electrodes as return electrodes;
receiving, from the user, an indication that at least one of the different magnitudes of current evoked muscle movement; and
determining, based on the indication, a calibration current for the first electrode,
wherein stimulating the first set of muscle fibers in the wrist by injecting current through the first electrode comprises injecting current at the level of the calibration current through the first electrode.

16. The method of claim 15, wherein the device comprises a user interface, and wherein receiving the indication comprises the user operating the user interface to receive the indication from the user.

17. The method of claim 14, wherein the method is a rehabilitative therapeutic method that further comprises, subsequent to stimulation of the first set of muscle fibers, providing an instruction to the user to voluntarily activate a muscle that comprises the first set of muscle fibers.

18. The method of claim 14, wherein the device is a prosthetic device, wherein the method further comprises detecting an intent of the user to activate a muscle that comprises the first set of muscle fibers, and wherein stimulating the first set of muscle fibers in the wrist by injecting current through the first electrode is performed in response to detecting the intent.

19. The method of claim 14, wherein the device is a therapeutic device, wherein the method further comprises detecting a phase of a tremor, and wherein stimulating the first set of muscle fibers in the wrist by injecting current through the first electrode is performed based on the detected phase such that the tremor is reduced.

20. (canceled)

21. The method of claim 20, wherein the output is a navigational direction, and wherein activating the muscle that comprises the first set of muscle fibers results in at least one of a finger or a hand of the user to move in a direction of the navigational direction.

22. The method of claim 20, wherein the output is indicative of the timing of onset of an act or event, and wherein activating the muscle that comprises the first set of muscle fibers results in the muscle moving with a timing that corresponds to the timing of onset of the act or event.

23. A non-transitory computer readable medium having stored thereon program instructions executable by at least one processor to cause the at least one processor to perform operations comprising:
stimulating a first set of muscle fibers in a wrist of a user by injecting current through a first electrode of a plurality of electrodes and using a first set of the plurality of electrodes as return electrodes, wherein the first set of electrodes comprises at least two electrodes, and wherein the plurality of electrodes are part of a device that is applied to the wrist of the user such that each electrode of the plurality of electrodes contacts the wrist at a respective different location around the wrist; and
stimulating a second set of muscle fibers in the wrist by injecting current through a second electrode of the plurality of electrodes and using a second set of the plurality of electrodes as return electrodes, wherein the second set of electrodes comprises at least two electrodes, and wherein the second electrode differs from the first electrode.

24. (canceled)
